(19) United States
(12) Patent Application Publication
(10) Pub. No.: US 2025/0340694 A1
(43) Pub. Date: Nov. 6, 2025

(54) METHOD OF PREPARING POLYURETHANE FOR BIOAPPLICATION

(71) Applicant: SKIN SOLUTION CO., LTD, Ansan-si (KR)

(72) Inventor: Yoo Sik LA, Suwon-si (KR)

(21) Appl. No.: 18/800,737

(22) Filed: Aug. 12, 2024

(30) Foreign Application Priority Data
May 3, 2024 (KR) 10-2024-0059298

(51) Int. Cl. C08G 18/66; C08G 18/28; C08G 18/32; C08G 18/48; C08G 18/73; C08G 18/75; C08G 18/76

(52) U.S. Cl. CPC C08G 18/6685; C08G 18/2865; C08G 18/3206; C08G 18/3234; C08G 18/4825; C08G 18/4854; C08G 18/6674; C08G 18/73; C08G 18/755; C08G 18/7671; C08G 18/7678

(57) ABSTRACT

A method of preparing a polyurethane for bioapplication, the method including dissolving a mixture of a polyester polyol and an extender in a reactor by heating to obtain a dissolved product, cooling the dissolved product to obtain a cooled product, and reacting a mixture of the cooled product and an isocyanate is disclosed.

### CROSS REFERENCE TO RELATED APPLICATION

The present application claims priority to Korean Patent Application No. 10-2024-0059298, filed May 3, 2024, the entire content of which is incorporated herein for all purposes by this reference.

### BACKGROUND

### 1. Field of the Disclosure

The present disclosure relates to a method of preparing a polyurethane for bioapplication and, specifically, to a method of preparing a polyurethane free of an N-nitroso-N-methyl-N-phenylamine derivative for bioapplication.

### 2. Description of the Related Art

Polyurethane, one of the six major synthetic polymers, is a family of plastics having excellent physical properties and a wide range of applications. Polyurethane collectively refers to a polymer compound containing urethane bonds formed by reacting polyols and isocyanates through polymerization reactions.

Polyurethane has been used as a material for a wide range of products, including daily necessities and consumer goods such as automobiles, building interiors, electronics, packaging, furniture, clothing, and the like.

In existing polyurethane preparation, polyester polyols, obtained by reacting adipic acid refined from petroleum with glycols, are reacted with isocyanates. However, such preparation processes involve various environmental pollutants and carcinogens, so the bioapplication (to the skin) thereof is limited.

Accordingly, there is a need for technologies to not only reduce environmental pollution but also prepare polyurethane free of carcinogens, such as N-nitroso-N-methyl-N-phenylamine derivatives, for bioapplication.

### Document of Related Art

### Patent Document

(Patent Document 1) Korean Patent No. 10-1824627 (published on Feb. 2, 2018)

### SUMMARY

The present disclosure aims to provide a method of preparing a polyurethane free of an N-nitroso-N-methyl-N-phenylamine derivative for bioapplication.

Additionally, the present disclosure aims to provide a polyurethane free of an N-nitroso-N-methyl-N-phenylamine derivative and thus applicable to bandages, dressings, pain relief patches, and the like for skin attachment without causing skin irritation.

In one embodiment, the present disclosure provides a method of preparing a polyurethane for bioapplication, the method including the following steps: (a) dissolving a mixture of a polyester polyol and an extender in a reactor by heating to obtain a dissolved product; (b) cooling the dissolved product to obtain a cooled product; and (c) reacting a mixture of the cooled product and an isocyanate.

Additionally, the polyol may be one or more selected from the group consisting of PEG 1000, PEG 2000, PEG 3000, PEG 4000, PPG 1000, PPG 2000, PPG 3000, PPG triol 3000, PPG 5000, PPG triol 5000, PPG 7000, PPG triol 7000, PTHF 1000, PTHF 2000, PTHF 3000, PTHF 5000, glycerin, sorbitol, pentaerythritol polycarbonate diol, polycarbonate diol, and poly(tetramethylene ether)glycol.

Additionally, the extender may be one or more selected from the group consisting of isophoronediamine (IPDA), dibutylamine (DBA), ethylene glycol (EG), butylene glycol (BG), neopentyl glycol (NPG), and 1,3-propanediol.

Additionally, in Step (a), the extender may be mixed in an amount in the range of 10 to 50 parts by weight based on 100 parts by weight of the polyester polyol.

Additionally, Step (a) may be performed at a temperature in the range of 80° C. to 100° C. for 5 to 30 minutes.

Additionally, the isocyanate may be one or more selected from the group consisting of isophorone diisocyanate (IPDI), hexamethylene diisocyanate (HDI), hydrogenated methylene diphenyl diisocyanate (HMDI), and naphthalene diisocyanate (NDI).

Additionally, in Step (c), the isocyanate may be included in an amount in the range of 50 to 150 parts by weight based on 100 parts by weight of the polyester polyol.

Additionally, Step (c) may be performed at a temperature in the range of 70° C. to 90° C. for 60 to 180 minutes.

In another embodiment, the present disclosure provides a polyurethane for bioapplication, the polyurethane prepared by the method described above.

In a further embodiment, the present disclosure provides an article for bioapplication, the article prepared using the polyurethane described above.

According to the present disclosure, provided can be a method of preparing a polyurethane free of an N-nitroso-N-methyl-N-phenylamine derivative for bioapplication.

Additionally, this polyurethane is N-nitroso-N-methyl-N-phenylamine derivative-free and thus applicable to many products such as bandages, tapes, dressings, and the like for bioapplication (to the skin).

### BRIEF DESCRIPTION OF THE DRAWINGS

FIG. 1 is a flowchart showing a method of preparing a polyurethane for bioapplication, according to one embodiment of the present disclosure;

FIG. 2A shows a structure of 1,6-hexamethylene diisocyanate (HDI);

FIG. 2B shows a structure of 4,4′-diisocyanato dicyclohexylmethane (hydrogenated MDI, H₁₂MDI);

FIG. 2C shows a structure of 4,4′-diisocyanato diphenylmethane (4,4′-MDI);

FIG. 2D shows a structure of 2,4′-diisocyanato diphenylmethane (2,4′-MDI);

FIG. 2E shows a structure of 2,2′-diisocyanato diphenylmethane (2,2′-MDI);

FIG. 2F shows a structure of 2,4-toluene diisocyanate (2,4-TDI);

FIG. 2G shows a structure of 2,6-toluene diisocyanate (2,6-TDI);

FIG. 2H shows a structure of naphthalene-1,5-diisocyanate (NDI); and

FIG. 2I shows a structure of 1-isocyanato-3-isocyanatomethyl-3,4,4-trimethyl-cyclohexane (isophorone diisocyanate, IPDI).

### DETAILED DESCRIPTION

In one embodiment, the present disclosure provides a method of preparing a polyurethane for bioapplication, the method including the following steps: (a) dissolving a mixture of a polyester polyol and an extender in a reactor by heating to obtain a dissolved product; (b) cooling the dissolved product to obtain a cooled product; and (c) reacting a mixture of the cooled product and an isocyanate.

Hereinbelow, embodiments of the present disclosure will be described in detail with reference to the attached drawings so that those skilled in the art can easily implement the present disclosure. However, the present disclosure may be implemented in many different forms and is not limited to the embodiments described herein. Throughout the specification, like reference numerals are used to identify like elements.

As used herein, the term “polyester polyol” refers to a polymer compound formed by the polymerization of ethylene glycol and adipic acid and containing a polyester group as a repeating unit in the main chain.

The polyurethane for bioapplication of the present disclosure may be prepared using a 2-liter double-jacketed glass reactor equipped with a cooler, a nitrogen seal tube, a stirrer, and a thermometer.

As used herein, the term “bioapplication” indicates “application to the skin”.

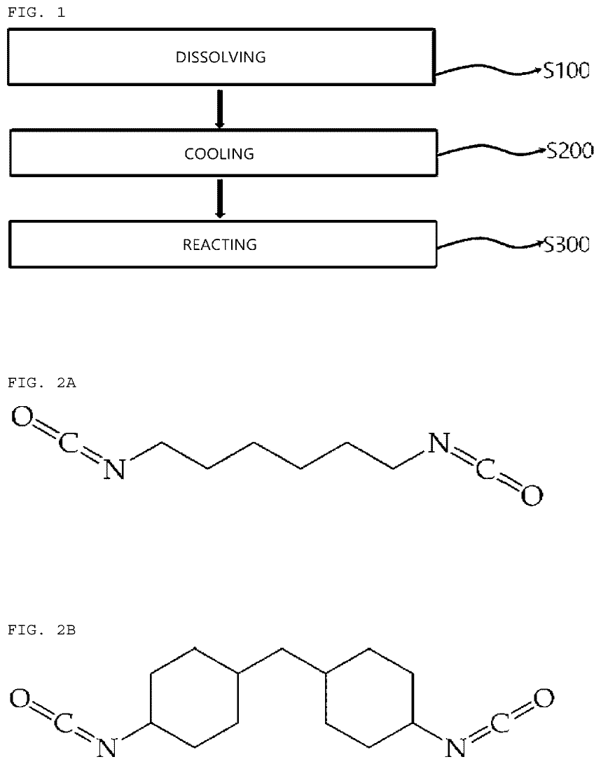

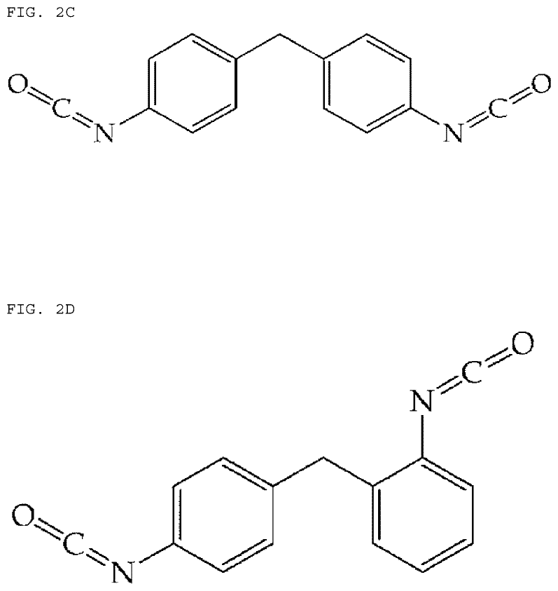

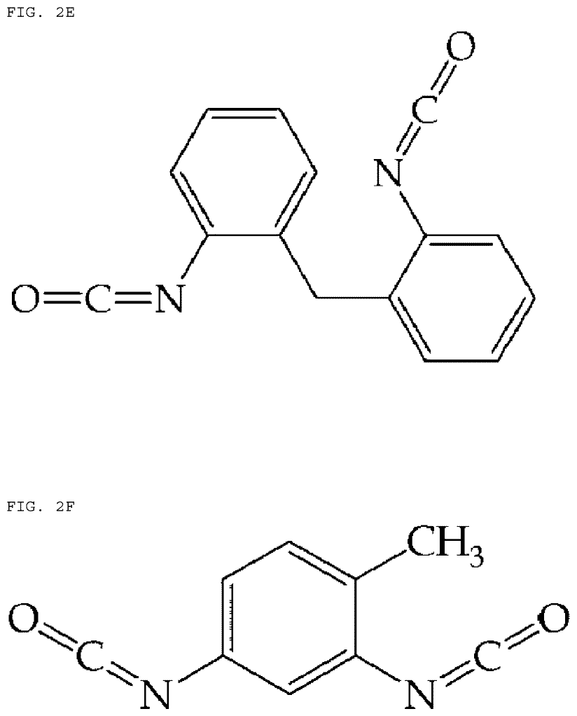

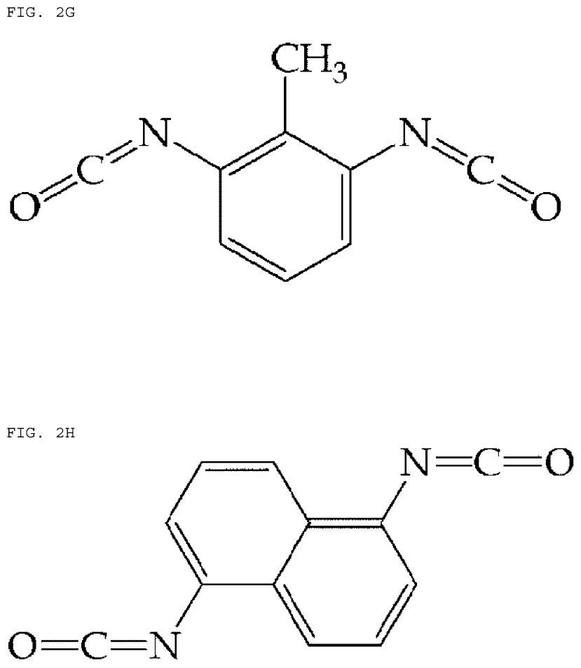

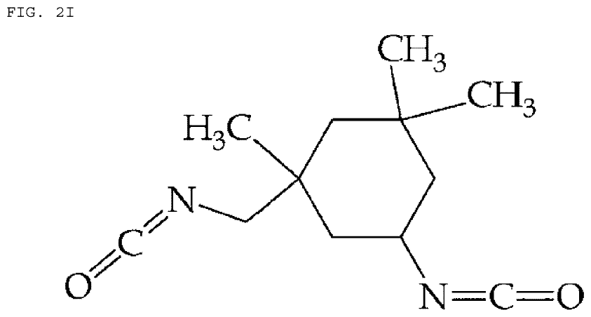

The present disclosure will be described in detail with reference to FIG. 1.

In one embodiment, the present disclosure provides a method of preparing a polyurethane for bioapplication, the method including the following steps: (a) dissolving a mixture of a polyester polyol and an extender in a reactor by heating to obtain a dissolved product (S100); (b) cooling the dissolved product to obtain a cooled product (S200); and (c) reacting a mixture of the cooled product and an isocyanate (S300).

### Dissolution Step (a)

First, as shown in FIG. 1, a step of dissolving a mixture of a polyester polyol and an extender in a reactor by heating (S100) is performed to obtain a dissolved product.

In Step (a), the chain of the polyester polyol is extended by mixing and reacting the extender with the polyester polyol.

The polyester polyol used in Step (a) is not limited as long as it is a polyester polyol used in the related art. However, the polyester polyol may specifically be one or more selected from the group consisting of glycerin, sorbitol, pentaerythritol polycarbonate diol, polycarbonate diol, and poly(tetramethylene ether)glycol. Additionally, the polyester polyol may also be commercially available under the trade names PEG 1000, PEG 2000, PEG 3000, PEG 4000, PPG 1000, PPG 2000, PPG 3000, PPG triol 3000, PPG 5000, PPG triol 5000, PPG 7000, PPG triol 7000, PTHF 1000, PTHF 2000, PTHF 3000, PTHF 5000, and the like.

The extender used in Step (a) is not limited as long as it extends the chain of the polyester polyol. However, the extender may specifically be one or more selected from the group consisting of isophoronediamine (IPDA), dibutylamine (DBA), ethylene glycol (EG), butylene glycol (BG), neopentyl glycol (NPG), and 1,3-propanediol and, more specifically, be isophoronediamine.

The extender may extend the chain of the polyester polyol and increase the molecular weight thereof.

In Step (a), the extender may be mixed in an amount in the range of 10 to 50 parts by weight based on 100 parts by weight of the polyester polyol. Specifically, the extender may be mixed in an amount in the range of 20 to 40 parts by weight based on 100 parts by weight of the polyester polyol, which is, more specifically, 30 parts by weight.

When the amount of the extender in Step (a) is less than 10 parts by weight, the effect of extending the chain of the polyester polyol may be minimal. On the contrary, when the amount of the extender exceeds 50 parts by weight, the amount of the polyol may be relatively low, leading to deterioration in the physical properties of the polyurethane to be ultimately prepared.

Step (a) may be performed at a temperature in the range of 80° C. to 100° C. for 5 to 30 minutes, specifically at a temperature in the range of 85° C. to 95° C. for 7 to 15 minutes, and more specifically at a temperature of 90° C. for 10 minutes.

When Step (a) is performed at a temperature lower than 80° C. for less than 5 minutes, dissolution may be incomplete, thus failing to prepare the polyurethane. On the contrary, when Step (a) is performed at a temperature exceeding 100° C. for more than 30 minutes, overreactions may occur due to high temperatures, resulting in discoloration or by-products.

### Cooling Step (b)

Next, as shown in FIG. 1, a step of cooling the dissolved product obtained through Step (a) (S200) is performed to obtain a cooled product.

The temperature of cooling the dissolved product obtained through Step (a) is not limited, but may be 65° C. or lower. Cooling may be performed up to the temperature, specifically, in the range of 40° C. to 65° C. and, more specifically, of 60° C. Additionally, the duration for preparing the cooled product by cooling the dissolved product obtained through Step (a) is not limited.

Step (b) may be performed by any method capable of cooling the dissolved product obtained through Step (a) so as to react with an isocyanate.

### Reaction Step (c)

Lastly, as shown in FIG. 1, Step (c) of reacting a mixture of the isocyanate and the cooled product obtained through Step (b) (S300) is performed.

In Step (c), the polyurethane may be prepared by mixing and reacting the cooled product obtained through Step (b) with the isocyanate.

In this case, the cooled product and a specific isocyanate may react, thus preparing a polyurethane free of an N-nitroso-N-methyl-N-phenylamine derivative for bioapplication.

In Step (c), the isocyanate may be one or more selected from the group consisting of isophorone diisocyanate (IPDI), hexamethylene diisocyanate (HDI), hydrogenated methylene diphenyl diisocyanate (HMDI), and naphthalene diisocyanate (NDI). Specifically, the isocyanate may be isophorone diisocyanate.

Additionally, in Step (c), the isocyanate may be included in an amount in the range of 50 to 150 parts by weight based on 100 parts by weight of the polyester polyol. Specifically, the isocyanate may be included in an amount in the range of 105 to 118 parts by weight based on 100 parts by weight of the polyester polyol, which is, more specifically, 115 parts by weight.

When the amount of the isocyanate does not fall within the above numerical range, the stoichiometric molar ratio between the polyester polyol and the isocyanate may be inappropriate, resulting in unreacted molar ratios and leading to deterioration in the quality of such a prepared polyurethane.

Additionally, Step (c) may be performed at a temperature in the range of 70° C. to 90° C. for 60 to 180 minutes, specifically at a temperature in the range of 75° C. to 85° C. for 90 to 150 minutes, and more specifically at a temperature of 80° C. for 120 minutes.

When Step (c) is performed at a temperature lower than 70° C. for less than 60 minutes, the polyester polyol and the isocyanate may fail to react perfectly. On the contrary, when Step (c) is performed at a temperature exceeding 90° C. for more than 180 minutes, overreactions may result in by-products.

In another embodiment, the present disclosure provides a polyurethane for bioapplication, the polyurethane prepared by the method described above.

Specifically, the present disclosure provides a polyurethane free f an N-nitroso-N-methyl-N-phenylamine derivate for bioapplication by synthesizing polyurethane using a specific isocyanate and polyester polyol.

In a further embodiment, the present disclosure provides an article for bioapplication, the article prepared using the polyurethane described above. For example, the article for bioapplication may be tapes, bandages, pain relief patches, dressings, facial masks, and the like for skin attachment.

Hereinbelow, the present disclosure will be described in more detail using examples. These examples are only for illustrating the present disclosure in more detail, and it is apparent to those skilled in the art that the scope of the present disclosure is not limited thereto.

### EXAMPLES

### Example 1

Into a 2-liter double-jacketed glass reactor equipped with a cooler, a nitrogen seal tube, a stirrer, and a thermometer, 100 parts by weight of a polyester polyol under the trade number PEG 1000, 15 parts by weight of isophoronediamine (IPDA), and 15 parts by weight of dibutylamine (DBA) were injected and then heated and dissolved at a temperature of 90° C. at 1000 rpm for 10 minutes. Next, the dissolved product was cooled to a temperature of 60° C. at 700 rpm, followed by injecting 115 parts by weight of isophorone diisocyanate (IPDI). Then, a reaction occurred at a temperature of 80° C. for 2 hours at 300 rpm, thus preparing a polyurethane with a viscosity of 7,000 cps for bioapplication.

### Example 2

A polyurethane for bioapplication was prepared in the same manner as in Example 1, except for using 115 parts by weight of hexamethylene diisocyanate (HDI) instead of 115 parts by weight of IPDI.

### Example 3

A polyurethane for bioapplication was prepared in the same manner as in Example 1, except for using 115 parts by weight of hydrogenated methylene diphenyl diisocyanate (HMDI) instead of 115 parts by weight of IPDI.

### Example 4

A polyurethane for bioapplication was prepared in the same manner as in Example 1, except for combining 40 parts by weight of the polyester polyol and 60 parts by weight of poly(tetramethylene ether)glycol (PTMEG) instead of 100 parts by weight of the polyester polyol.

### Example 5

A polyurethane for bioapplication was prepared in the same manner as in Example 1, except for using 30 parts by weight of ethylene glycol (EG) instead of 15 parts by weight of IPDA and 15 parts by weight of DBA.

### Example 6

A polyurethane for bioapplication was prepared in the same manner as in Example 1, except for using 30 parts by weight of butylene glycol (BG) instead of 15 parts by weight of IPDA and 15 parts by weight of DBA.

### Example 7

A polyurethane for bioapplication was prepared in the same manner as in Example 1, except for using 30 parts by weight of neopentyl glycol (NPG) instead of 15 parts by weight of IPDA and 15 parts by weight of DBA.

### Example 8

A polyurethane for bioapplication was prepared in the same manner as in Example 1, except for using 115 parts by weight of naphthalene diisocyanate (NDI) instead of 115 parts by weight of IPDI.

### Example 9

A polyurethane for bioapplication was prepared in the same manner as in Example 1, except for using 30 parts by weight of 1,3-propanediol instead of 15 parts by weight of IPDA and 15 parts by weight of DBA.

### COMPARATIVE EXAMPLES

### Comparative Example 1

A polyurethane for bioapplication was prepared in the same manner as in Example 1, except for using 115 parts by weight of toluene diisocyanate (TDI) instead of IPDI.

### Comparative Example 2

A polyurethane for bioapplication was prepared in the same manner as in Example 1, except for using 115 parts by weight of methylene diphenyl diisocyanate (MDI) instead of IPDI.

### Comparative Example 3

A polyurethane for bioapplication was prepared in the same manner as in Example 1, except for using 50 parts by weight of TDI and 60 parts by weight of MDI instead of 115 parts by weight of IPDI.

### Test Examples

Whether N-nitroso-N-methyl-N-phenylamine derivatives were detected in the polyurethanes prepared according to Examples 1 to 8 and Comparative Examples 1 to 3 were examined.

Test method: About 1 to 5 g of each sample was measured and placed in a conical flask. Then, an artificial saliva test solution preheated at 40±2° C. was added in a volume equivalent to about ten times the weight of the sample (about 10 mL of the artificial saliva test solution per 1 g of the sample).

Next, 4.2 g of sodium bicarbonate, 0.5 g of sodium chloride, 0.2 g of potassium carbonate, and 0.03 g of sodium nitrite were prepared, dissolved in 950 mL of distilled water, and set to a pH of 9 using a 0.1 mol/L NaOH solution, followed by adding distilled water to a total volume of 1,000 mL. The sample was sufficiently submerged in the solution and, after closing the stopper, then left to stand at 40±2° C. for 60 minutes, followed by taking 1 mL of the extracted solution into a vial for liquid chromatography (LC) analysis and adding 0.02 mL of an internal standard substance. Subsequently, 5 mL of the extracted solution was transferred to a 20 mL vial, and 0.5 mL of 0.1 M hydrochloric acid solution was added. Then, after closing the stopper, the vial was shaken to be mixed well and left to stand at 40±2° C. for 30 minutes. Afterward, the stopper was opened to add and mix 1 mL of 1 M sodium hydroxide solution well.

After taking 1 mL of the test solution into the vial for LC analysis, 0.02 mL of the internal standard substance was added and analyzed by liquid chromatography-tandem mass spectrometry (LC-MS/MS).

The analysis was performed using an Agilent 6470 Triple Quad LC/MS, C18 (Rapid Resolution High Definition (RRHD) 2.1 mm Inner Diameter (I.D.)×100 mm, 1.8 μm) column under the following analysis conditions: a flow rate of 0.3 mL/min and a column temperature of 30° C. The mobile phases were A: 0.1% formic acid in diluted water and B: 0.1% formic acid in acetonitrile, and the analysis was performed in a multiple reaction monitoring (MRM) mode under A:B gradient conditions (70:30→10:90→-70:30). The analysis results thereof are shown in Table 1 below.

TABLE 1

| | | | | | | | | | | | Compar- | Compar- | Compar- |
|---|---|---|---|---|---|---|---|---|---|---|---|---|---|
| | | | | | | | | | | | ative | ative | ative |
| | | Exam- | Exam- | Exam- | Exam- | Exam- | Exam- | Exam- | Exam- | Exam- | Exam- | Exam- | Exam- |
| Classification | | ple 1 | ple 2 | ple 3 | ple 4 | ple 5 | ple 6 | ple 7 | ple 8 | ple 9 | ple 1 | ple 2 | ple 3 |
| Polyure- | Isocy- | IPDI | HDI | HMDI | IPDI | IPDI | IPDI | IPDI | NDI | NDI | TDI | MDI | TDI/ |
| thane | anate | | | | | | | | | | | | MDI |
| compo- | | | | | | | | | | | | | |
| nent | | | | | | | | | | | | | |
| | Polyol | Ester | Ester | Ester | Ester | Ester | Ester | Ester | Ester | Ester | Ester | Ester | Ester |
| | | polyol | polyol | polyol | polyol/ | polyol | polyol | polyol | polyol | polyol | polyol | polyol | polyol |
| | | | | | PTMEG | | | | | | | | |
| | Ex- | IPDA/ | IPDA/ | IPDA/ | IPDA/ | EG | BG | NPG | IPDA/ | 1.3 | IPDA/ | IPDA/ | IPDA/ |
| | tender | DBA | DBA | DBA | DBA | | | | DBA | propanediol | DBA | DBA | DBA |

| | | | | | | | | | | | | |
|---|---|---|---|---|---|---|---|---|---|---|---|---|
| Results | Unde- | Unde- | Unde- | Unde- | Unde- | Unde- | Unde- | Unde- | Undetected | Detected | Detected | Detected |
| | tected | tected | tected | tected | tected | tected | tected | tected | | | | |

As can be seen in Table 1, it is confirmed that N-nitroso-N-methyl-N-phenylamine derivatives are detected in all Comparative Examples 1 to 3 but are undetected in all Examples when comparing Example 3 with 1 to 8. Additionally, Comparative Example 2, it is confirmed that N-nitroso-N-methyl-N-phenylamine derivatives are detected in Comparative Example 2 using MDI but are undetected in Example 3 using hydrogenated MDI. In other words, it is confirmed that there is also a difference depending on whether hydrogenation is involved or not.

In summary, in the present disclosure, the polyurethane is prepared using a specific isocyanate, thus of N-nitroso-N-methyl-N-enabling a polyurethane free phenylamine derivatives for bioapplication to be prepared. Additionally, such a prepared polyurethane may be applied to various products such as bandages, tapes, dressings, and the like for bioapplication (to the skin).

According to the present disclosure, this polyurethane is N-nitroso-N-methyl-N-phenylamine derivative-free and thus applicable to many products such as bandages, tapes, dressings, and the like for bioapplication (to the skin).

While a particular embodiment of the present method of preparing a polyurethane for bioapplication has been described herein, it will be appreciated by those skilled in the art that changes and modifications may be made thereto without departing from the invention in its broader aspects and as set forth in the following claims.

What is claimed is:

1. A method of preparing a polyurethane for bioapplication, the method comprising:
(a) dissolving a mixture of a polyester polyol and an extender in a reactor by heating to obtain a dissolved product;
(b) cooling the dissolved product to obtain a cooled product; and
(c) reacting a mixture of the cooled product and an isocyanate.

2. The method of claim 1, wherein the polyol is one or more selected from the group consisting of PEG 1000, PEG 2000, PEG 3000, PEG 4000, PPG 1000, PPG 2000, PPG 3000, PPG triol 3000, PPG 5000, PPG triol 5000, PPG 7000, PPG triol 7000, PTHF 1000, PTHF 2000, PTHF 3000, PTHF 5000, glycerin, sorbitol, pentaerythritol polycarbonate diol, polycarbonate diol, and poly(tetramethylene ether)glycol.

3. The method of claim 1, wherein the extender is one or more selected from the group consisting of isophoronediamine (IPDA), dibutylamine (DBA), ethylene glycol (EG), butylene glycol (BG), neopentyl glycol (NPG), and 1,3-propanediol.

4. The method of claim 1, wherein in the (a) dissolving, the extender is mixed in an amount in a range of 10 to 50 parts by weight based on 100 parts by weight of the polyester polyol.

5. The method of claim 1, wherein the (a) dissolving is performed at a temperature in a range of 80° C. to 100° C. for 5 to 30 minutes.

6. The method of claim 1, wherein the isocyanate is one or more selected from the group consisting of isophorone diisocyanate (IPDI), hexamethylene diisocyanate (HDI), hydrogenated methylene diphenyl diisocyanate (HMDI), and naphthalene diisocyanate (NDI).

7. The method of claim 1, wherein in the (c) reacting, the isocyanate is included in an amount in a range of 50 to 150 parts by weight based on 100 parts by weight of the polyester polyol.

8. The method of claim 1, wherein the (c) reacting is performed at a temperature in a range of 70° C. to 90° C. for 60 to 180 minutes.

9. A polyurethane for bioapplication, the polyurethane prepared by the method of claim 1.

10. An article for bioapplication, the article prepared using the polyurethane of claim 9.
